(19) United States
(12) Patent Application Publication
(10) Pub. No.: US 2002/0165529 A1
(43) Pub. Date: Nov. 7, 2002

(54) Method and apparatus for non-invasive energy delivery

(76) Inventor: Christopher James Danek, Santa Clara, CA (US)

Correspondence Address: LARRY S. ZELSON, 2011 LIVINGSTON STREET, ALLENTOWN, PA, 18104, US

(21) Appl. No.: 10/116,443

(22) Filed: Apr. 4, 2002

Related U.S. Application Data
(60) Provisional application No. 60/282,298, filed on Apr. 5, 2001.

(51) Int. Cl. A61B 18/18

(52) U.S. Cl. 606/28; 606/32; 606/41; 607/101

(57) ABSTRACT

Systems and methods for selectively applying energy to a target location on an external body surface for therapeutic purpose, such as removal of body hair, shrinkage of collagen, coagulation of blood vessels, and treatment of lesions. The present invention applies various sources of energy, including radiofrequency, ultrasound, and microwave, to modify subcutaneous tissue while prevent damage to surface tissue. The frequency and intensity of the energy delivery is modulated based upon feedback temperature measurements, present algorithms, user selected algorithms, or user visual cues.

### CROSS-REFERENCE TO RELATED APPLICATIONS

This application claims the benefit of the filing date of provisional application No. 60/282298, filed on Apr. 6, 2001.

### FEDERALLY SPONSORED RESEARCH

Not Applicable

### SEQUENCE LISTING OR PROGRAM

Not Applicable

### BACKGROUND OF INVENTION

1. Field of Invention

This invention relates to devices and methods for delivering energy to localized areas of the surface of the human body, and more particularly to devices which are capable of delivering energy in the form of radiofrequency, ultrasound, or microwave at desired energy frequencies and intensities for therapeutic purpose.

2. Description of Related Art

The present invention includes methods and apparatus for non-invasive energy delivery below the tissue surface to achieve desired changes in targeted tissue while minimizing collateral damage to adjacent and surface tissues not targeted for treatment. While there are existing light-based methods—such as certain lasers and flashlamps—that offer similar advantages, some of the methods and apparatus in this invention may be used to improve those light-based approaches to non-invasive energy delivery below the tissue surface.

The present invention includes methods and apparatus that do not rely on light energy. Potential applications include, but are not limited to, the removal of body hair for cosmetic or medical purposes, shrinking of collagen for cosmetic or medical purposes, including but not limited to wrinkle removal; structural remodeling, the coagulation of blood vessels near the tissue surface, and treatment of lesions.

There is a large demand for the cosmetic and medical procedures as described. This patent application describes methods and apparatus that offer the following advantages: (a) persistence of therapeutic effect, such as hair loss, collagen remodeling, vessel closure, (b) a non-invasive approach that does not require penetrating the tissue surface, and (c) absence of disfiguring side-effect such as visible scar tissue formation.

The target in producing persistent hair loss is the follicle. The target in wrinkle reduction is sub-surface collagen. The target in eliminating spider veins is subsurface blood vessels. The therapeutic target may vary, but in each of the applications described, the object is to deliver sufficient energy so that the target sustains a temperature-time history that effects the desired change, while minimizing collateral damage to adjacent tissue structures, in particular the surface tissue. This desired change can be produced by mechanical energy, thermal energy (heat or cold), radiofrequency energy, microwave energy, ultrasound energy, or chemical means. This invention focuses on methods and apparatus for energy delivery that result in heating or cooling of the target tissue structure while protecting nearby tissue.

### SUMMARY OF THE INVENTION

The treatment system that is the subject of this invention includes an energy delivery device and an energy source. The energy delivery device guides energy supplied by the source to the targeted tissue. The delivery device may be made for single use (disposable) or made to be reusable (able to be cleaned and re-sterilized if necessary). The delivery device may alternatively have a reusable component designed to connect the energy source to a disposable energy delivery element.

Energy Delivery to Tissue

There are various means of delivering energy to the tissue to achieve the desired result of target modification and minimal collateral damage. The non-light means included as part of this invention include radiofrequency (RF) energy delivery, ultrasound (US) energy delivery, microwave energy delivery, and cryogenic cooling. The first three result in heating of tissue, and is believed to be most effective when operating in the temperature range of 50° C. to 100° C. The optimum temperature depends on the properties of the targeted tissue, the surrounding tissue structure properties, and the duration of treatment.

Radiofrequency energy may be delivered in monopolar or bipolar mode. In monopolar mode a return electrode must be placed on the patient. If desired, its location may be chosen based on the area to be treated. For example, the return electrode could be placed opposite the region being treated. An example of this would be placement on the back of the patient's shoulder when treating the front of the shoulder. In the case of treating the face, the return electrode could be a mouthpiece inserted in the patient's mouth, or a nasal insert.

There are a wide variety of configurations for the active electrodes in either monopolar or bipolar configurations. The material may be chosen to allow conduction of RF current with minimal heating of the electrode (high conductivity), or to allow conduction of RF energy with a deliberate heating of the electrode (low conductivity). They may be flat or curved to promote uniform contact over the electrode surface. The contact area of the active electrodes may be round (circular, elliptical) or rectilinear (square, rectangular, polygonal)—virtually any shape is possible. The shape may be chosen, for example, to suit the anatomy to be treated or to allow optimal coverage for repeated activations (for example, a hexagon shape offers the advantage of providing complete coverage when treating irregular areas through multiple activations). In bipolar mode, the active electrodes can be configured on opposite sides of graspers (such as a forceps or tweezer configuration), to allow current to pass directly through tissue grasped in the device. The number of electrodes may be varied to allow patterned delivery of energy to tissue; at least one active electrode for monopolar and at least two active electrodes for bipolar are required. Multiple electrodes can be configured in many different patterns such as circular patterns, radial patterns, rectangular arrays, or in approximation of any of the shapes described in this application. Use of multiple electrodes allows the incorporation of other features within the working area of the device such as cooling elements or suction ports.

Ultrasound energy can be delivery via an ultrasound transmitter. The ultrasound transmitter can be positioned in acoustic contact with the tissue surface (via mechanical contact or acoustic coupling via gel, for example). Ultrasound energy can be delivered to subsurface tissue. The penetration of the ultrasound depends upon the frequency chosen. These frequencies are well known from the ultrasound sonography and echocardiography fields. The extent of damage also depends on ultrasound intensity (or amplitude). Ultrasound may be delivered through optically clear structures used as viewing windows to observe surface tissue during treatment.

By positioning two or more ultrasound delivery elements in an array so their resulting output constructively interferes, the zone where energy delivery exceeds the therapeutic threshold may be controlled, and focused in a subsurface location.

Adding ultrasound transduction will allow sensing of, for example, blood flow. This is useful when the target structure is a blood vessel. It is also possible to detect changes in tissue properties by using pulsed ultrasound. The tissue damage zone size and location may be tailored by suitable choices in ultrasound delivery (frequency, intensity) and in the size, number, and positioning (location and aim) of ultrasound delivery elements. All of these factors may be made adjustable by the user.

Microwave energy can be delivered by means of a shielded antenna placed in proximity to the tissue surface under treatment. The design of the antenna controls the radiation patterns into the tissue. A guard that prevents unintended microwave radiation in the backward or lateral directions can be incorporated in the device for safety.

Cryogenic contact cooling can be used to drop the temperature of the targeted tissue structure below a damage threshold. This could be useful in hair removal. Long pulses of cooling mixed with no cooling or short pulses of heating could be used to do subsurface damage while protecting the surface.

Protection of Surface Layers

All of the energy delivery forms described in this application can be applied in steady (continuous) or transient fashion. For transient delivery, energy can be pulsed or delivered in a waveform modulated with a carrier wave such as a sinusoid or train of square pulses. The parameters of transient energy delivery (such as duty cycle and amplitude) can be chosen in such a way to achieve the desired time-temperature history of targeted structures but allow collateral tissue structures to relax to temperatures (by bio-heat transfer mechanisms such as perfusion or conduction) that are outside the window where permanent change occurs.

The energy delivery pattern (steady, transient, and all the parameters described herein) may be made adjustable by the user in response to visual cues or clinical indication. It may also be varied automatically in response to feedback from sensors such as temperature, pressure, or flow sensing elements built into the device.

Protection of surface layers can be achieved through passive means, such as the transient energy delivery described in this application, or through active means. A contact probe may be used to cool the surface (in the case of RF, US, microwave). The cooling may be either steady, at a level that serves to protect the surface and immediately adjacent tissue, or transient and synchronized with the delivery of therapeutic energy. In the case of cryogenic treatment, a heating probe may be used instead to achieve the same goal. The contact probe could be a thermoelectric element configured to provide either heating or cooling as required. Protection may also be achieved by directing a flow of gas or liquid against the tissue surface. The temperature and physical properties of the stream of gas or liquid (including velocity, viscosity, and specific heat) may be chosen to provide optimum protection.

The contact probe could be applied either before or after treatment as a separate device. It could also be built into the treatment device to allow simultaneous or synchronized protection. This configuration is especially convenient when the energy delivery device is either a small single element or configured as an array (which allows placement of protection elements within or around the array).

Energy Source

The invention comprises an energy source (such as an RF generator, microwave generator, or other energy source) in conjunction with a device for delivering energy to the tissue. The energy source can have one or more performance enhancing features. For example, the source may be configured with a microprocessor control unit to allow delivery of energy according to a preset algorithm. Energy may be delivered with a pre-defined profile (intensity versus time) or the energy delivery parameters may be made user adjustable. The energy delivery may be controlled via a feedback loop using a sensor (for example, temperature, pressure, or flow). The energy controller may have a fixed coefficients or the controller coefficients may be varied adaptively depending upon the sensed tissue response to energy delivery. Safety algorithms may be employed for example to limit energy delivery or to limit sensed tissue temperature. These algorithms could shut off energy delivery or modulate the energy delivery.

The energy source may be powered by AC electric power or DC power, such as from batteries. The source may be configured to mount in an instrument rack, be placed on a counter or table, or clamp to a holder such as an IV pole.

### BRIEF DESCRIPTION OF THE FIGURES

FIG. 1 is a schematic overview of the treatment system.

FIGS. 2A-2B illustrate monopolar radiofrequency electrode configuration examples.

FIG. 2C illustrates a radiofrequency electrode for monopolar or bipolar energy delivery.

FIG. 3 illustrates the application of a bipolar radiofrequency electrode configuration to tissue treatment.

FIG. 4 illustrates the application of an ultrasound transmitter configuration to tissue treatment.

FIG. 5 illustrates the application of a shielded microwave antenna to tissue treatment.

### DETAILED DESCRIPTION

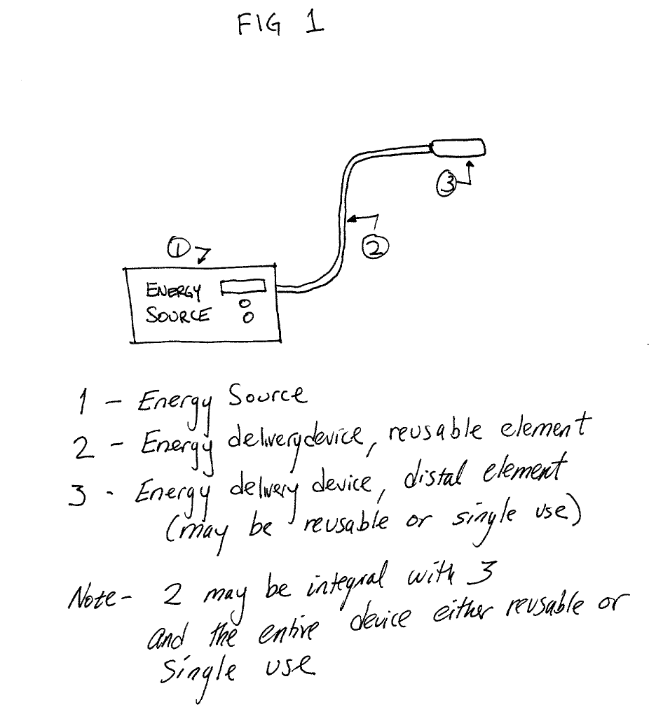

An embodiment of this invention is the combination is illustrated in FIG. 1 as an energy source 1, an energy transfer conduit 2, and an energy delivery probe 3. The conduit may be integrated into the probe and need not be a separate element in the system.

The energy source 1 incorporates the possibility of multiple energy generators, including radiofrequency, ultrasound, and microwave. Energy output can be configured to follow a profile of intensity versus time based upon either pre-defined parameters or user input. Measurement of skin temperature, by thermocouple, thermister, or optical means, may be used in conjunction with closed-loop control of the energy output. Feedback control of the temperature of the skin under treatment or of the energy delivery element is used to adaptively vary the energy output. For example, if the sensed temperature is insufficient to achieve the desire therapeutic effects, then energy output will be increased. Likewise, if the sensed temperature is so high as to be in danger of causing undesired tissue damage, the energy output will be decreased. The most effective range of temperature control is believed to be between 50° C. and 100° C. The adaptive control feature can use accumulated knowledge to improve the accuracy of the energy delivery parameters based on historical performance. While the first described embodiment utilizes radiofrequency as the energy source, the microprocessor control strategies employed are equally transferable to a device using ultrasound or microwave energy, and could be employed in a similar manner to an energy sink such as a source of cryogenic cooling.

The energy transfer conduit 2 is a capable of carrying the energy source in use, including radiofrequency, ultrasound, and microwave. This conduit is also capable of carrying signals, including but not limited to measured temperature, from the probe back to the energy source. In the energy sink case, the energy transfer conduit would incorporate a tube carrying cryogenic fluid.

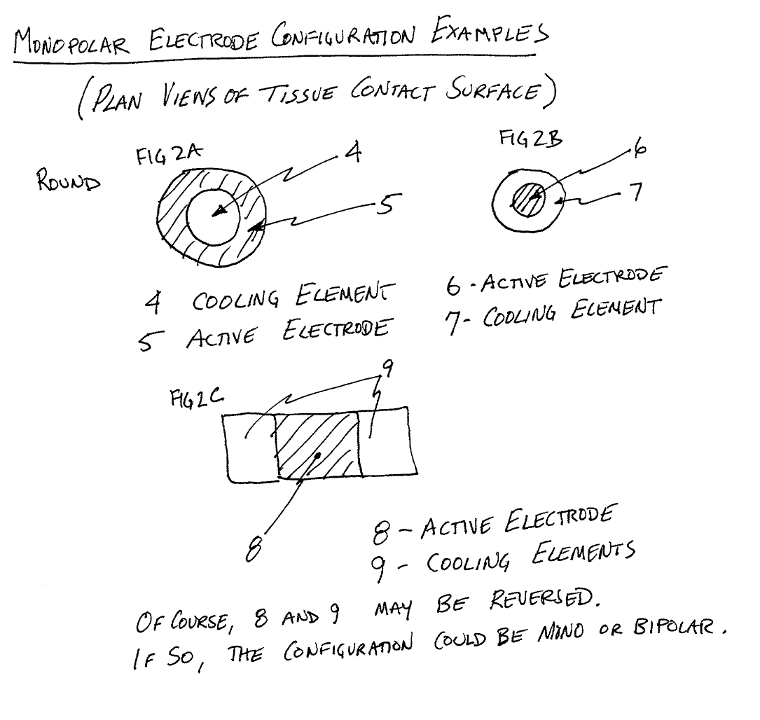

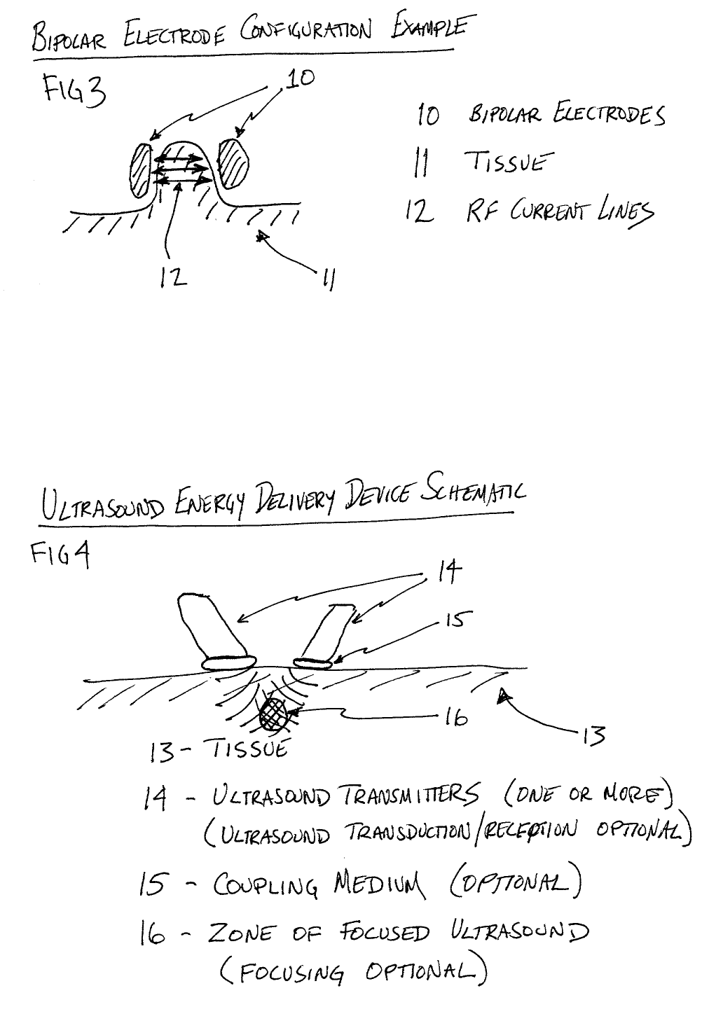

The RF energy delivery probe 3 is shown in further detail in FIGS. 2A, 2B, and 2C. The energy delivery probe incorporates an active electrode and a cooling element. The tip of the energy delivery element can be in multiple geometric configurations. In the basic embodiment of FIG. 2A, a round cooling element 4 is surrounded by an annular monopolar RF electrode 5. In another embodiment, as shown in FIG. 2B, a round monopolar RF electrode 6 is surrounded by an annular cooling element 7. The embodiment of FIG. 2C shows bipolar RF electrodes 9 separated by cooling element 8. This configuration would function equally well as a monopolar RF electrode if the elements are reversed such that the monopolar electrode 8 is flanked by cooling elements 9.

An application of a bipolar RF electrode configuration is shown in FIG. 3, where the bipolar RF electrodes 10 are positioned such that current lines of the RF energy pass through the tissue being treated.

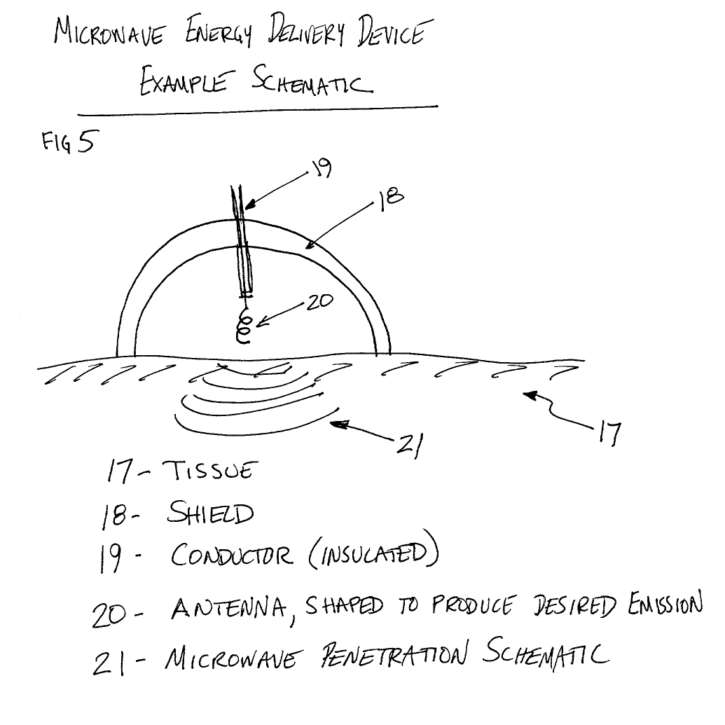

An application of an ultrasound transmitter configuration is shown in FIG. 4. Ultrasound transmitters 14 are positioned on the surface of the tissue being treated, with or without the use of a coupling medium 15. One or more ultrasound transmitters may be used. When multiple transmitters are used, the transmitted energy can be focused particularly on the region under treatment.

An application of a microwave energy delivery device configuration is shown in FIG. 5. The microwave antenna 20, shaped to produce the desired emission, is fed microwave energy via an insulated conductor 19. A microwave shield 18 is positioned and shaped so as to allow microwave energy to interact only with the tissue under treatment and to prevent any microwave radiation from affecting surrounding tissue or the operator of the device.

1. A method of treating subcutaneous tissue to achieve a therapeutic effect of hair removal, collagen shrinkage, vessel closure, or lesion ablation, without damaging the surface layer of tissue and without physically penetrating the surface layer of tissue, comprising:
transferring energy to or from the tissue with a probe connected to an energy source by a flexible elongate means.

2. The method of claim 1, further comprising:
maintaining said probe in a static position during energy transfer; and
repositioning said probe as desired to cover additional areas.

3. The method of claim 2, wherein said energy source comprises:
an energy generator capable of generating microwave, ultrasound, or radiofrequency energy; and
a microprocessor controller capable of adjusting the frequency and the intensity of the energy output.

4. The method of claim 3, wherein said probe further comprises:
a temperature sensing element.

5. The method of claim 4, wherein said probe further comprises:
an active heating or cooling means for protecting the surface tissue from damage by controlling the surface tissue temperature.

6. The method of claim 5, wherein said active heating or cooling means is a thermoelectric element.

7. The method of claim 6, wherein said probe comprises:
an array of one or more ultrasound transmitting transducers configured to produce a subcutaneous pattern of ultrasound.

8. The method of claim 5, further comprising:
modulating energy output of said energy source based upon feedback from said temperature sensing element.

9. The method of claim 8, wherein the tissue being treated is maintained at a target temperature in the range of about 50° C. to about 100° C.

10. The method of claim 9, where the sensor is a thermocouple or thermistor.

11. The method of claim 9, wherein said temperature sensing element is an optical sensor.

12. The method of claim 3, further comprising:
modulating the energy delivery manually, according to visual indicators of tissue effect.

13. An apparatus for directing energy to an epidermal surface for therapeutic purpose, comprising:
an energy transfer probe with the distal end being an atraumatic tissue contact surface
an energy source; and
a flexible elongate means for transmitting energy and electronic signals to or from said energy source to a connector on the proximal end of said probe.

14. The apparatus of claim 13, the distal end of said probe further comprising a temperature sensing element.

15. The apparatus of claim 14, wherein said temperature sensing element is a thermocouple or thermistor.

16. The apparatus of claim 14, wherein said temperature sensing element is an optical sensor.

17. The apparatus of claim 14, the distal end of said probe further comprising an active heating or cooling means for protecting the surface tissue from damage by controlling the surface tissue temperature.

18. The apparatus of claim 17, wherein said active heating or cooling means is a thermoelectric element.

19. The apparatus of claim 13, the distal end of said probe further comprising:
an array of one or more ultrasound transmitting transducers configured to produce a subcutaneous pattern of ultrasound.

20. The apparatus of claim 19, the distal end of said probe further comprising:
an array of one or more ultrasound receiving transducers configured to sense subcutaneous tissue effect or blood flow.

21. The apparatus of claim 13, the distal end of said probe further comprising an array of one or more ultrasound dual function transducers, wherein each transducer a transmitting portion configured to produce a subcutaneous effect and a receiving portion configured to sense subcutaneous tissue effect or blood flow.

22. The apparatus of claim 19, said energy source comprising:
an ultrasound generator capable of modulating the frequency and the intensity of the ultrasound energy delivered to said transducers; and
a means to control, independently or collectively, the frequency and the intensity of the ultrasound energy delivered to each said transducer.

23. The apparatus of claim 20, said energy source comprising:
an ultrasound generator capable of modulating the frequency and the intensity of the ultrasound energy delivered to said transducers; and
a means to control, independently or collectively, the frequency and the intensity of the ultrasound energy delivered to each said transducer.

24. The apparatus of claim 21, said energy source comprising:
an ultrasound generator capable of modulating the frequency and the intensity of the ultrasound energy delivered to said transducers; and
a means to control, independently or collectively, the frequency and the intensity of the ultrasound energy delivered to each said transducer.

25. The apparatus of claim 13, further comprising:
a flexible elongate member, said member having one or more conduit means for transmitting fluid or providing suction from said energy source to said probe.

26. The apparatus of claim 13, said energy source comprising a microwave generator.

27. The apparatus of claim 26, the distal end of said probe further comprising:
one or more microwave transmitting elements; and
a shield around each said microwave element which prevents microwave energy transmission in a backward or lateral direction away from the cutaneous region targeted for therapeutic treatment.

28. The apparatus of claim 13, said energy source comprising a radiofrequency generator.

29. The apparatus of claim 13, the distal end of said probe further comprising one or more radiofrequency transmitting elements.

30. The apparatus of claim 14, said energy source comprising:
an energy generator capable of generating microwave, ultrasound, or radiofrequency energy; and
a microprocessor controller capable of adjusting the frequency and the intensity of the energy output.
